(19) United States
(12) Patent Application Publication
(10) Pub. No.: US 2003/0096192 A1
(43) Pub. Date: May 22, 2003

(54) Optical data carrier comprising a xanthene dye as light-absorbent compound in the information layer

(76) Inventors: Horst Berneth, Leverkusen (DE); Friedrich-Karl Bruder, Krefeld (DE); Wilfried Haese, Odenthal (DE); Rainer Hagen, Leverkusen (DE); Karin Hassenruck, Dusseldorf (DE); Serguei Kostromine, Swisttal (DE); Peter Landenberger, Koln (DE); Rafael Oser, Krefeld (DE); Thomas Sommermann, Bergisch Gladbach (DE); Josef-Walter Stawitz, Odenthal (DE); Thomas Bieringer, Odenthal (DE)

Correspondence Address: BAYER CORPORATION, PATENT DEPARTMENT, 100 BAYER ROAD, PITTSBURGH, PA, 15205, US

(21) Appl. No.: 10/102,588

(22) Filed: Mar. 20, 2002

(30) Foreign Application Priority Data
Mar. 28, 2001 (DE) 10115227.2
Apr. 6, 2001 (DE) 10117462.4
Jul. 25, 2001 (DE) 10136064.9
Jul. 25, 2001 (DE) 10136063.0
Jan. 24, 2002 (DE) 10202571.1

(51) Int. Cl. G11B 7/24; C07D 311/88

(52) U.S. Cl. 430/270.15; 549/226; 549/227; 428/64.8; 428/913; 430/945; 430/270.19

(57) ABSTRACT

Optical data carrier comprising a preferably transparent substrate which may, if desired, have previously been coated with one or more reflection layers and to whose surface a light-writeable information layer, if desired one or more reflection layers and, if desired, a protective layer or a further substrate or a covering layer have been applied, which can be written on or read by means of blue or red light, preferably laser light, where the information layer comprises a light-absorbent compound and, if desired, a binder, characterized in that at least one xanthene dye which contains at least two anionic groups and has at least one cation containing at least one conjugated π system having at least 6 π electrons as counterion, where the cation must not be benzyltrimethylammonium, benzyltriethylammonium, tetraphenylphosphonium, butyltriphenylphosphonium or ethyltriphenylphosphonium, is used as light-absorbent compound.

The invention relates to a write-once optical data carrier comprising a xanthene dye as light-absorbent compound in the information layer, to a process for its production and also to the application of the above-mentioned dyes to a polymer substrate, in particular polycarbonate, by spin coating or vapour deposition.

Write-once optical data carriers using specific light-absorbent substances or mixtures thereof are particularly suitable for use in high-density writeable optical data stores which operate with blue laser diodes, in particular GaN or SHG laser diodes (360-460 nm), and/or for use in DVD-R or CD-R disks which operate with red (635-660 nm) or infrared (780-830 nm) laser diodes.

The write-once compact disk (CD-R, 780 nm) has recently experienced enormous volume growth and represents the technically established system.

The next generation of optical data stores—DVDs—is currently being introduced onto the market. Through the use of shorter-wavelength laser radiation (635-660 nm) and higher numerical aperture NA, the storage density can be increased. The writeable format in this case is DVD-R.

Today, optical data storage formats which use blue laser diodes (based on GaN, JP-A 08 191 171 or Second Harmonic Generation SHG JP-A 09 050 629) (360 nm-460 nm) with high laser power are being developed. Writeable optical data stores will therefore also be used in this generation. The achievable storage density depends on the focussing of the laser spot on the information plane. Spot size scales with the laser wavelength λ/NA. NA is the numerical aperture of the objective lens used. In order to obtain the highest possible storage density, the use of the smallest possible wavelength λ is the aim. At present, 390 nm is possible on the basis of semiconductor laser diodes.

The patent literature describes dye-based writeable optical data stores which are equally suitable for CD-R and DVD-R systems (JP-A 11 043 481 and JP-A 10 181 206). To achieve a high reflectivity and a high modulation height of the read-out signal and also to achieve sufficient sensitivity in writing, use is made of the fact that the IR wavelength of 780 nm of CD-Rs is located at the foot of the long wavelength flank of the absorption peak of the dye and the red wavelength of 635 nm or 650 nm of DVD-Rs is located at the foot of the short wavelength flank of the absorption peak of the dye. In JP-A 02 557 335, JP-A 10 058 828, JP-A 06 336 086, JP-A 02 865 955, WO-A 09 917 284 and U.S. Pat. No. 5,266,699, this concept is extended to the 450 nm working wavelength region on the short wavelength flank and the red and IR region on the long wavelength flank of the absorption peak.

Apart from the abovementioned optical properties, the writeable information layer comprising light-absorbent organic substances has to have a substantially amorphous morphology to keep the noise signal during writing or reading as small as possible. For this reason, it is particularly preferred that crystallization of the light-absorbent substances be prevented in the application of the substances by spin coating from a solution, by vapour deposition and/or sublimation during subsequent covering with metallic or dielectric layers under reduced pressure.

The amorphous layer comprising light-absorbent substances preferably has a high heat distortion resistance, since otherwise further layers of organic or inorganic material which are applied to the light-absorbent information layer by sputtering or vapour deposition would form blurred boundaries due to diffusion and thus adversely affect the reflectivity. Furthermore, a light-absorbent substance which has insufficient heat distortion resistance can, at the boundary to a polymeric support, diffuse into the latter and once again adversely affect the reflectivity.

A light-absorbent substance whose vapour pressure is too high can sublime during the abovementioned deposition of further layers by sputtering or vapour deposition in a high vacuum and thus reduce the layer thickness to below the desired value. This in turn has an adverse effect on the reflectivity.

It is therefore an object of the invention to provide suitable compounds which satisfy the high requirements (e.g. light stability, favourable signal/noise ratio, damage-free application to the substrate material, and the like) for use in the information layer in a write-once optical data carrier, in particular for high-density writeable optical data storage formats in a laser wavelength range from 340 to 680 nm.

An optical storage medium having a high capacity and comprising xanthene dyes is known from EP-A 0 805 441. The xanthene dyes claimed can have up to four positive or negative excess charges and corresponding counterions. Cationic counterions described are protons and metal, ammonium or phosphonium cations.

Surprisingly, it has been found that light-absorbent compounds selected from the group of xanthene dyes which contain at least two anionic groups and have at least one cation containing at least one particular conjugated a system having at least 6× electrons as counterion can satisfy the abovementioned requirement profile particularly well.

The invention accordingly provides an optical data carrier comprising a preferably transparent substrate which may, if desired, have previously been coated with one or more reflection layers and to whose surface a light-writeable information layer, if desired one or more reflection layers and, if desired, a protective layer or a further substrate or a covering layer have been applied, which can be written on or read by means of blue or red light, preferably laser light, where the information layer comprises a light-absorbent compound and, if desired, a binder, characterized in that at least one xanthene dye which contains at least two anionic groups and has at least one cation containing at least one conjugated π system having at least 6 π electrons as counterion, with the proviso that the cation is not benzyltrimethylammonium, benzyltriethylammonium, tetraphenylphosphonium, butyltriphenylphosphonium or ethyltriphenylphosphonium, is used as light-absorbent compound.

The light-absorbent compound should preferably be able to be changed thermally. The thermal change preferably occurs at a temperature of <600° C., particularly preferably at a temperature of <400° C., very particularly preferably at a temperature of <300° C., in particular <200° C. Such a change can be, for example, a decomposition or chemical change of the chromophoric centre of the light-absorbent compound.

Preference is given to a xanthene dye of the formula (I)

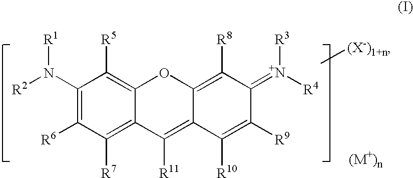

where

R¹ to R⁴ represent, independently of one another, hydrogen, C₁-C₁₆-alkyl, C₃-C₆-alkenyl, C₅-C₇-cycloalkyl, C₇-C₁₆-aralkyl, C₆-C₁₀-aryl or a heterocyclic radical, which may be substituted by nonionic radicals or an anionic group X or

NR¹R² or NR³R⁴ represent, independently of one another, a five- or six-membered saturated ring which is bound via N and may additionally contain an N or O atom and/or be substituted by nonionic radicals,

R⁵ to R¹⁰ represent, independently of one another, hydrogen, halogen, C₁-C₁₆-alkyl, C₁-C₁₆-alkoxy, C₁-C₁₆-alkylthio, cyano or nitro or

R¹;R⁵, R²;R⁶, R³;R⁸ or R⁴;R⁹ represent, independently of one another, a two- or three-membered bridge which may contain an N or O atom and/or be substituted by nonionic radicals,

R¹¹ represents hydrogen, C₁-C₁₆-alkyl, C₅-C₇-cycloalkyl, C₆-C₁₀-aryl or a heterocyclic radical which may be substituted by nonionic radicals or an anionic group X⁻,

X⁻ represents an anionic group of the formula —COO^{—}, —SO₃⁻ or —O—SO₃⁻ or one equivalent of a dianionic group of the formula —PO₃²⁻ or —O—PO₃²⁻,

M⁺ represents a cation or one equivalent of a polycation which contains at least one conjugated π system having at least 6 π electrons, and

n represents an integer from 1 to 3,

with the proviso that M⁺ does not represent benzyltrimethylammonium, benzyltrimethylammonium, tetraphenylphosphonium, butyltriphenylphosphonium or ethyltriphenylphosphonium.

Possible nonionic radicals are, for example, C₁-C₄-alkyl, C₁-C₄-alkoxy, halogen, cyano, nitro, C₁-C₄-alkoxycarbonyl, C₁-C₄-alkylthio, C₁-C₄-alkanoylamino, benzoylamino, mono- or di-C₁-C₄-alkylamino.

Alkyl radicals including those in alkoxy, alkylthio or aralkyl groups and also those mentioned later in the text may be straight-chain or branched.

Heterocyclic radicals are furyl, thienyl, pyridyl or a radical of the formula

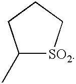

Alkyl, alkoxy, aryl and heterocyclic radicals including those which are mentioned later in the text may, if desired, bear further radicals such as alkyl, halogen, nitro, cyano, CO—NH₂, alkoxy, trialkylsilyl, trialkylsiloxy or phenyl, the alkyl and alkoxy radicals may be straight-chain or branched, the alkyl radicals may be partially halogenated or perhalogenated, the alkyl and alkoxy radicals may be ethoxylated or propoxylated or silylated, adjacent alkyl and/or alkoxy radicals on aryl or heterocyclic radicals may together form a three- or four-membered bridge and the heterocyclic radicals may be benzo-fused and/or quaternized.

For the purposes of the present invention, a cation or one equivalent of a polycation, which contains at least one conjugated π system having at least 6 π electrons, is preferably

a) an aromatically or heteroaromatically substituted ammonium, sulphonium or iodonium salt,

b) a cyclic onium salt,

c) a redox system in its oxidized cationic or radical-cationic form or

d) a cationic dye system.

Examples of these are:

a) anilinium salts, diphenyliodonium, thien-2-yl-trimethylammonium,

b) pyridinium salts, quinolinium salts, benzothiazolium salts, dithiolium salts,

c) bipyridinium salts, quinodiimmonium salts, metallocenyls such as ferrocenyl (Fe^{III}(C₅H₅)₂⁺), manganocenyl (Mn^{III}(CO)₃C₅H₅⁺),

d) cationic organic dyes.

For the purposes of the present invention, aromatic and heteroaromatic substituents are, for example: phenyl, tolyl, anisyl, chlorophenyl, naphthyl, luryl, thienyl, pyridyl, quinolyl.

Cationic organic dyes can, for example, come from the classes of cyanines, streptocyanines, hemicyanines, diazahemicyanines, nullmethines, enamine dyes, hydrazone dyes, di- or tri(het)arylmethane dyes, xanthene dyes, azine dyes (phenazines, oxazines, thiazines) or, for example, come from the classes of azo dyes, anthraquinone dyes, neutrocyanines, porphyrins or phthalocyanines if they bear at least one localized positive charge. Such dyes are known, for example, from H. Bemeth, Cationic Dyes in Ullmann's Encyclopedia of Industrial Chemistry, VCH, 6^{th} edition.

Preference is given to cationic organic dyes whose λₘₐₓ differs from the λₘₐₓ₂ of the xanthese dyes by not more than 50 nm, preferably not more than 30 nm, very particularly preferably not more than 10 nm.

Redox systems are known, for example, from S. Hünig, H. Berneth, Topics in Current Chemistry, vol. 92, 1, 1980 and K. Deuchert, S. Hünig, Angew. Chem. 1978, 90, 927. Cations M⁺ which are suitable for the purposes of the invention are in each case the oxidation state OX and the free-radical state SEM, as long as they are cationic.

Particular preference is given to xanthene dyes of the formula (I) in which

R¹ to R⁴ represent, independently of one another, hydrogen, methyl, ethyl, propyl, butyl, chloroethyl, cyanoethyl, hydroxyethyl, hydroxypropyl, —CH₂CH₂COO^{—}, —CH₂CH₂CH₂COO⁻, —CH₂CH₂CH₂CH₂COO^{—}, —CH₂CH₂SO₃⁻, —CH₂CH₂CH₂SO₃⁻, —CH₂CH₂CH₂CH₂SO₃⁻, —CH₂CH₂OSO₃⁻, allyl, cyclopentyl, cyclohexyl, benzyl, phenethyl, phenyl, tolyl, anisyl, —C₆H₄—SO₃⁻, pyridyl or furyl or

NR¹R² or NR³R⁴ represent, independently of one another, pyrrolidino, piperidino, morpholino, piperazino or N-methylpiperazino,

R⁵ to R¹⁰ represent, independently of one another, hydrogen, chlorine, methyl or methoxy or

R¹;R⁵, R²;R⁶, R³;R⁸ or R⁴;R⁹ represent, independently of one another, a —CH₂CH₂—, —CH₂CH₂CH₂— or —CH₂CH₂—O— bridge,

R¹¹ represents hydrogen, —CH₂CH₂COO^{—}, —CH₂CH₂CH₂COO^{—}, —CH₂CH₂CH₂CH₂COO^{—}, —CH₂CH₂SO₃⁻, —CH₂CH₂CH₂SO₃⁻, —CH₂CH₂CH₂CH₂SO₃⁻, —CH₂CH₂OSO₃⁻, phenyl, naphthyl or pyridyl which are substituted by up to two —COO^{—}, —SO₃⁻, CN, —COO-methyl to -butyl radicals,

where the radicals R¹ to R⁴ and R¹¹ contain a total of at least two —COO⁻ or —SO₃⁻ groups,

M⁺ represents a cation or one equivalent of a polycation of one of the following formulae,

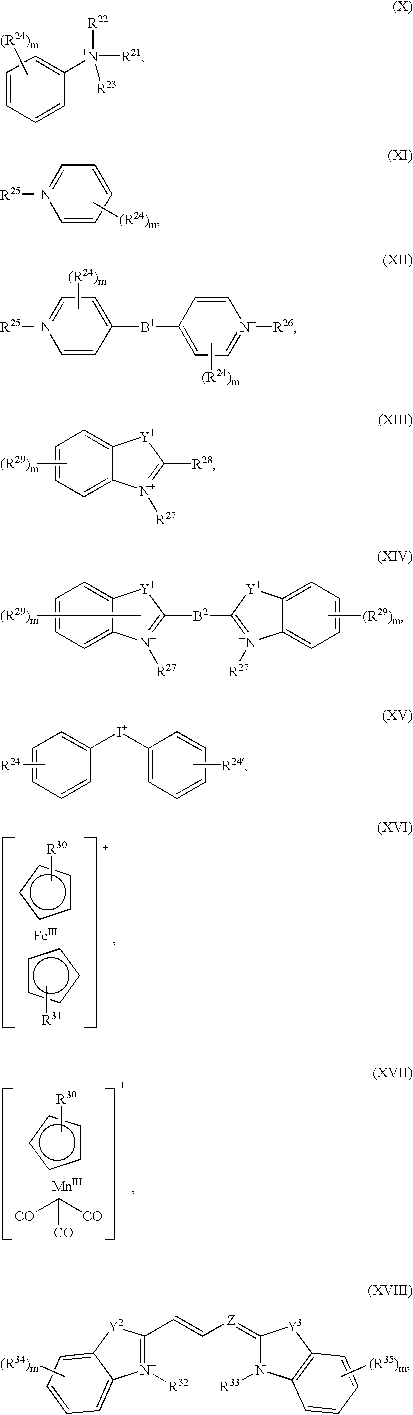

where

R²¹ to R²³, R³⁶, R³⁷, R³⁹ to R⁴², R⁵¹ to R⁵⁴, R⁵⁷, R⁶¹ to R⁶⁶, R⁷², R⁷³, R^{72′}, R^{73′}, R⁷⁶, R⁷⁷, R⁸⁰ and R⁸¹ represent, independently of one another, hydrogen, C₁-C₁₆-alkyl, C₃-C₆-alkenyl, C₅-C₇-cycloalkyl, C₇-C₁₆-aralkyl or C₆-C₁₀-aryl which may be substituted by nonionic radicals or

two adjacent radicals together with the nitrogen atom connecting them represent, independently of one another, a five- or six-membered saturated ring which is bound via N and may additionally contain an N or O atom and/or be substituted by nonionic radicals,

R²⁵ to R²⁷, R³², R³³ and R⁷⁸ represent, independently of one another, C₁-C₁₆-alkyl, C₃-C₆-alkenyl, C₅-C₇-cycloalkyl, C₇-C₁₆-aralkyl or C₆-C₁₀-aryl which may be substituted by nonionic radicals,

R²⁸ represents hydrogen, chlorine, amino, C₁-C₁₆-alkyl, C₃-C₆-alkenyl, C₅-C₇-cycloalkyl, C₇-C₁₆-aralkyl or C₆-C₁₆-aryl,

R²⁴, R^{24′}, R²⁹ to R³¹, R³⁴, R³⁵ and R⁷⁹ represent, independently of one another, hydrogen, halogen, C₁-C₈-alkyl, C₁-C₈-alkoxy, C₁-C₄-alkylthio, cyano or nitro or

two adjacent radicals R²⁴, R²⁹, R³⁴ and R³⁵ represent a —CH═CH—CH═CH-bridge,

R³⁸, R⁵⁵ and R⁵⁶ represent, independently of one another, hydrogen, halogen, C₁-C₄-alkyl, C₁-C₄-alkoxy, cyano, nitro, C₁-C₄-alkoxycarbonyl, C₁-C₄-alkanoylamino or C₁-C₄-alkanesulphonylamino and R³⁸ together with R³⁶ may form a —(CH₂)₂— or —(CH₂)₃— bridge,

R⁴³ to R⁴⁸, R⁶⁰, R⁶⁷, R⁶⁸ and R⁸² represent, independently of one another, hydrogen, halogen, C₁-C₈-alkyl, C₁-C₈-alkoxy or C₁-C₄-alkylthio and R⁴³ together with R³⁹, R⁴⁴ together with R⁴⁰, R⁴⁶ together with R⁴¹, R⁴⁷ together with R⁴¹, R⁶⁷ together with R⁶³, R⁶⁸ together with R⁶⁵ and R⁸² together with R⁸⁰ may form a —(CH₂)₂— or —CH₂)₃— bridge,

R⁴⁹, R⁷⁴ and R^{74′} represent, independently of one another, hydrogen, C₁-C₁₆-alkyl, C₅-C₇-cycloalkyl or C₆-C₁₀-aryl which may be substituted by nonionic radicals,

Y¹ to Y³ represent, independently of one another, O, S, NR⁵⁷, CR⁵⁸R⁵⁹ or —CH═CH—,

Y⁴ represents CR⁶⁰ or N,

Y⁵ and Y⁶ represent, independently of one another, O, S, NR⁵⁷ or CR⁵⁸R⁵⁹,

Z, Y⁷ and Y^{7′} represent, independently of one another, N, CH or C—CN,

Y⁸ and Y^{8′} represent, independently of one another, O or S,

R⁵⁸ and R⁵⁹ represent, independently of one another, hydrogen or C₁-C₄-alkyl or

CR⁵⁸R⁵⁹ represents a ring of the formula

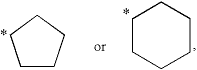

where two single bonds go out from the asterisked (*) atom,

R⁵⁰ represents hydrogen, halogen, C₁-C₄-alkyl, C₁-C₄-alkoxy, C₁-C₄-alkylthio, cyano, nitro, C₁-C₄-alkoxycarbonyl, mono- or dialkylamino, pyrrolidino, piperidino or morpholino or

R⁵⁰; R⁶⁰ form a —CH═CH—CH═CH— bridge,

R⁶⁹ and R⁷⁵ represent, independently of one another, hydrogen, C₁-C₄-alkyl or a radical of the formula

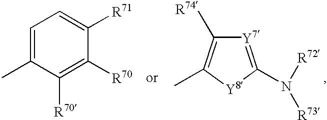

R⁷⁰ and R^{70′} represent, independently of one another, hydrogen, halogen, C₁-C₈-alkyl, C₁-C₈-alkoxy or C₁-C₄-alkylthio or together form a —CH═CH—CH═CH-bridge or R⁷⁰ together with R⁷⁷ may form a —(CH₂)₂— or —(CH₂)₃— bridge,

R⁷¹ represents hydrogen, halogen, C₁-C₈-alkyl, C₁-C₈-alkoxy, C₁-C₄-alkylthio, mono- or di-C₁-C₈-alkylamino, anilino or N—C₁-C₈-alkyl-anilino,

A represents a radical of the formula

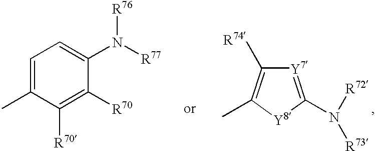

B¹ represents a direct bond, —CH═CH— or —C≡C—,

B² represents a direct bond, —CH═CH—, —C≡C— or thien-2,5-diyl,

Het represents a five- or six-membered aromatic or pseudoaromatic heterocyclic ring which contains from 1 to 3 heteroatoms selected from the group consisting of N, O and S and may be benzo-fused and/or substituted by up to three nonionic radicals,

m represents an integer from 1 to 3, where, if m>1, the radicals indexed by m may have different meanings and

n represents an integer from 1 to 2.

Examples of heterocyclic rings Het are thiazolyl, benzothiazolyl, thienyl, benzothienyl, pyrazolyl, thiadiazolyl, pyridyl.

Likewise suitable as M⁺ is the radical-cationic oxidation state SEM corresponding to the indication of the formula (XXV) and having the formula

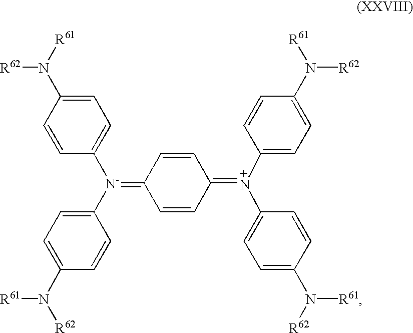

where R⁶¹ and R⁶² are as defined above.

In a particularly preferred embodiment, the xanthene dyes used are ones of the formula (II)

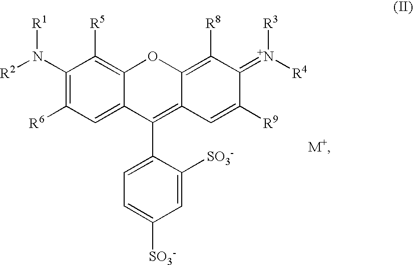

where

R¹ to R⁴ represent, independently of one another, hydrogen, methyl, ethyl, propyl, butyl, cyanoethyl, hydroxyethyl, hydroxypropyl, cyclohexyl, benzyl or phenyl or

NR¹R² or NR³R⁴ represent, independently of one another, pyrrolidino, piperidino or morpholino,

R⁵, R⁶, R⁸ and R⁹ represent, independently of one another, hydrogen, methyl or methoxy or

R¹;R⁵, R²;R⁶, R³;R⁸ or R⁴;R⁹ represent, independently of one another, a —CH₂CH₂CH₂— bridge,

M⁺ is a cation or one equivalent of a polycation of one of the formulae (X) to (XII), (XV), (XVI), (XVIII) to (XX), (XXIV), (XXVI), (XXVII) or (XXVIII),

where

R²¹ to R²³, R³⁶, R³⁷, R³⁹ to R⁴², R⁵⁷, R⁶¹ to R⁶⁶, R⁷², R⁷³, R^{72′}, R^{73′}, R⁷⁶, R⁷⁷, R⁸⁰ and R⁸¹ represent, independently of one another, hydrogen, methyl, ethyl, propyl, butyl, cyanoethyl, hydroxyethyl, hydroxypropyl, cyclohexyl, benzyl or phenyl or

NR²¹R²², NR³⁶R³⁷ NR³⁹R⁴⁰, NR⁴¹R⁴², NR⁶¹R⁶² and NR⁸⁰R⁸¹ represent, independently of one another, pyrrolidino, piperidino or morpholino,

R²⁵ to R²⁷, R³², R³³ and R⁷⁸ represent, independently of one another, methyl, ethyl, propyl, butyl, cyanoethyl, hydroxyethyl, hydroxypropyl, cyclohexyl or benzyl,

R²⁴, R^{24′}, R³⁴, R³⁵ and R⁷⁹ represent, independently of one another, hydrogen, chlorine, methyl, methoxy, cyano or nitro or

two adjacent radicals R²⁴, R³⁴ or R³⁵ represent a —CH═CH—CH═CH— bridge,

R³⁰ and R³¹ are identical and represent methyl, ethyl, propyl, 2-propyl, butyl or tert-butyl,

R³⁸ represents hydrogen, chlorine, methyl, methoxy, cyano, nitro, methoxycarbonyl, acetylamino or methanesulphonylamino,

R⁴³ to R⁴⁸, R⁶⁷, R⁶⁸ and R⁸² represent, independently of one another, hydrogen, chlorine, methyl or methoxy,

R⁴⁹, R⁷⁴and R^{74′} represent, independently of one another, methyl, cyclohexyl or phenyl,

B¹ represents a direct bond,

Y² and Y³ are identical and represent O, S, NR⁶⁷, CR⁵⁸R⁵⁹ or —CH═CH—,

Y⁶ represents O, S or NR⁵⁷,

R⁵⁸ and R⁵⁹ are identical and represent methyl,

Z, Y⁷ and Y^{7′} represent CH,

Y⁸ and Y⁸ represent O or S and are identical,

R⁶⁹ represents hydrogen or a radical of the formula

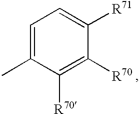

R⁷⁵ represents hydrogen or a radical of the formula

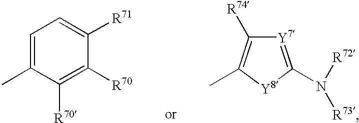

R⁷⁰ and R^{70′} represent, independently of one another, hydrogen, chlorine, methyl or methoxy or together form a —CH═CH—CH═CH— bridge,

R⁷¹ represents hydrogen, chlorine, methyl, methoxy, ethoxy, dimethylamino, diethylamino, N-methyl-N-cyanoethylamino, N-methyl-N-hydroxyethylamino, anilino or N-methyl-anilino,

A represents a radical of the formula

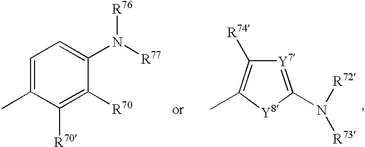

m represents an integer from 1 to 3, where, if m>1, the radicals indexed by m may have different meanings.

In a very particularly preferred embodiment, the xanthene dyes used are ones of the formula (II-A)

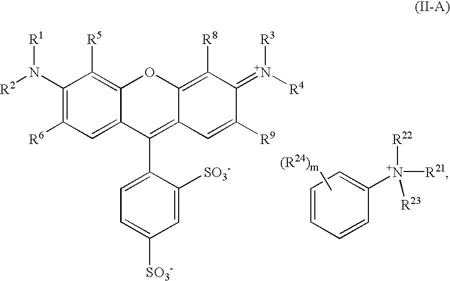

where

R¹ to R⁴ and R²¹ to R²³ represent, independently of one another, hydrogen, methyl, ethyl, propyl or butyl or

NR¹R², NR³R⁴ or NR²¹R²² represent, independently of one another, pyrrolidino, piperidino or morpholino,

R⁵, R⁶, R⁸ and R⁹ represent, independently of one another, hydrogen or methyl or

R¹;R⁵, R²;R⁶, R³;R⁸ or R⁴;R⁹ represent, independently of one another, a —CH₂CH₂CH₂— bridge,

R²⁴ represents hydrogen, methyl or methoxy or two adjacent radicals R²⁴ represent a —CH═CH—CH═CH— bridge and

m represents 1 or 2.

In a likewise very particularly preferred embodiment, the xanthene dyes used are ones of the formula (II-B)

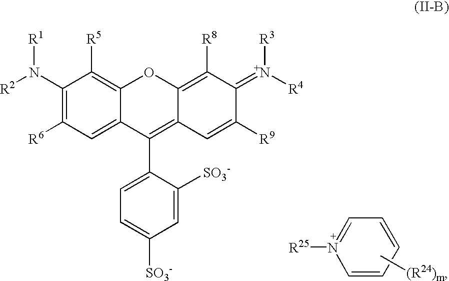

where

R¹ to R⁴ and R²⁵ represent, independently of one another, hydrogen, methyl, ethyl, propyl or butyl or

NR¹R² or NR³R⁴ represent, independently of one another, pyrrolidino, piperidino or morpholino,

R⁵, R⁶, R⁸ and R⁹ represent, independently of one another, hydrogen or methyl or

R¹;R⁵, R²;R⁶, R³;R⁸ or R⁴;R⁹ represent, independently of one another, a —CH₂CH₂CH₂— bridge,

R²⁴ represents hydrogen, methyl or methoxy or two adjacent radicals R²⁴ represent a —CH═CH—CH═CH— bridge and

m represents 1 or 2.

In a likewise very particularly preferred embodiment, the xanthene dyes used are ones of the formula (II-C)

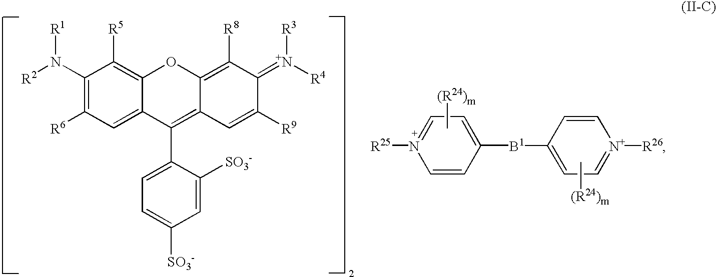

where

R¹ to R⁴, R²⁵ and R²⁶ represent, independently of one another, hydrogen, methyl, ethyl, propyl or butyl or

NR¹R² or NR³R⁴ represent, independently of one another, pyrrolidino, piperidino or morpholino,

R⁵, R⁶, R⁸ and R⁹ represent, independently of one another, hydrogen or methyl or

R¹;R⁵, R²;R⁶, R³;R⁸ or R⁴;R? represent, independently of one another, a —CH₂CH₂CH₂— bridge,

B¹ represents a direct bond,

R²⁴ represents hydrogen, methyl or methoxy or two adjacent radicals R²⁴ represent a —CH═CH—CH═CH— bridge and

m represents 1 or 2.

In a likewise very particularly preferred embodiment, the xanthene dyes used are ones of the formula (II-D)

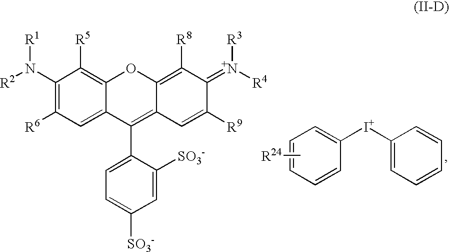

where

R¹ to R⁴ represent, independently of one another, hydrogen, methyl, ethyl, propyl or butyl or

NR¹R² or NR³R⁴ represent, independently of one another, pyrrolidino, piperidino or morpholino,

R⁵, R⁶, R⁸ and R⁹ represent, independently of one another, hydrogen or methyl or

R¹;R⁵, R²;R⁶, R³;R⁸ or R⁴;R⁹ represent, independently of one another, a —CH₂CH₂CH₂— bridge and

R²⁴ represents hydrogen, methoxy, ethoxy, butoxy or octoxy.

In a likewise very particularly preferred embodiment, the xanthene dyes used are ones of the formula (II-E)

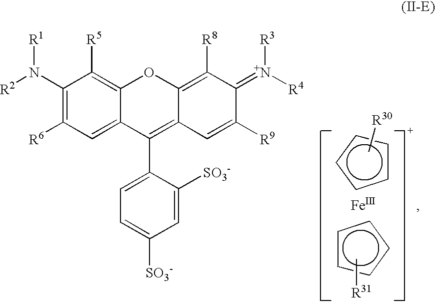

where

R¹ to R⁴ represent, independently of one another, hydrogen, methyl, ethyl, propyl or butyl or

NR¹R² or NR³R⁴ represent, independently of one another, pyrrolidino, piperidino or morpholino,

R⁵, R⁶, R⁸ and R⁹ represent, independently of one another, hydrogen or methyl or

R¹;R⁵, R²;R⁶, R³;R⁸ or R⁴;R⁹ represent, independently of one another, a —CH₂CH₂CH₂— bridge and

R³⁰ and R³¹ are identical and represent hydrogen, methyl or tert-butyl.

In a likewise very particularly preferred embodiment, the xanthene dyes used are ones of the formula (I-F)

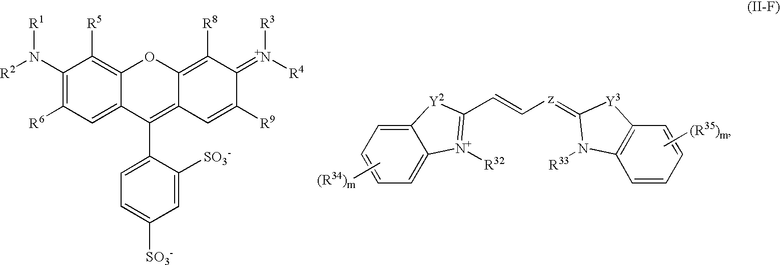

where

R¹ to R⁴, R³² and R³³ represent, independently of one another, hydrogen, methyl, ethyl, propyl or butyl or

NR¹R² or NR³R⁴ represent, independently of one another, pyrrolidino, piperidino or morpholino,

R⁵, R⁶, R⁸ and R⁹ represent, independently of one another, hydrogen or methyl or

R¹;R⁵, R²;R⁶, R³;R⁸ or R⁴;R⁹ represent, independently of one another, a —CH₂CH₂CH₂— bridge and

R³⁴ and R³⁵ represent, independently of one another, hydrogen, methyl, methoxy or methoxycarbonyl or two adjacent radicals represent a —CH═CH—CH═CH-bridge,

m represents 1 or 2,

Y² and Y³ represent, independently of one another, O, S, C(CH₃)₂ or —CH═CH— and

z represents CH.

Y² is preferably S or C(CH₃)₂ and Y³ is preferably —CH═CH—.

In a likewise very particularly preferred embodiment, the xanthene dyes used are ones of the formula (II-G)

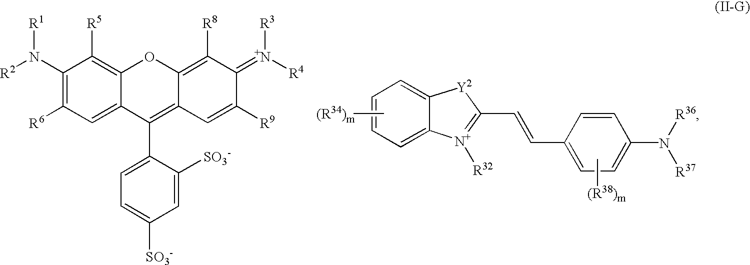

where

R¹ to R⁴, R³², R³⁶ and R²⁷ represent, independently of one another, hydrogen, methyl, ethyl, propyl or butyl or

NR¹R², NR³R⁴ and NR³⁶R³⁷ represent, independently of one another, pyrrolidino, piperidino or morpholino and

R³⁶ may also represent phenyl, methoxyphenyl or ethoxyphenyl,

R⁵, R⁶, R⁸ and R⁹ represent, independently of one another, hydrogen or methyl or

R¹;R⁵, R⁵;R⁶, R³;R⁸ or R⁴;R⁹ represent, independently of one another, a —CH₂CH₂CH₂— bridge and

R³⁴ represents hydrogen, methyl, methoxy or methoxycarbonyl,

R³⁸ represents hydrogen, methyl, methoxy, cyano, acetylamino or methanesulphonylamino,

m represents 1 and

Y² represents O, S, C(CH₃)₂ or —CH═CH—.

In a likewise very particularly preferred embodiment, the xanthene dyes used are ones of the formula (II-H)

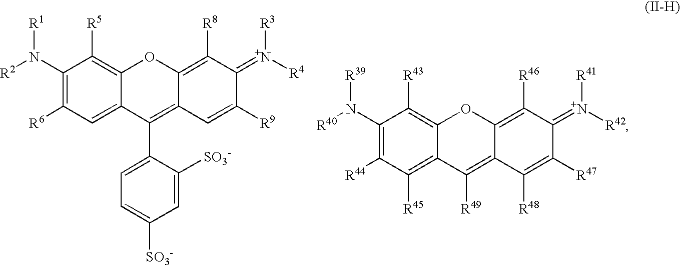

where

R¹ to R⁴ and R³⁹ to R⁴² represent, independently of one another, hydrogen, methyl, ethyl, propyl or butyl or

NR¹R², NR³R⁴, NR³⁹R⁴⁰ and NR⁴¹R⁴² represent, independently of one another, pyrrolidino, piperidino or morpholino,

R⁵, R⁶, R⁸, R⁹, R⁴³, R⁴⁴, R⁴⁶ and R⁴⁷ represent, independently of one another, hydrogen or methyl or

R¹;R⁵, R²;R⁶, R³;R⁸, R⁴;R⁹, R³⁹;R⁴³, R⁴⁰;R⁴⁴, R⁴¹;R⁴⁶ and R⁴²;R⁴⁷ represent, independently of one another, a —CH₂CH₂CH₂— bridge,

R⁴⁵ and R⁴⁸ represent hydrogen and

R⁴⁹ represents hydrogen, methyl or phenyl.

In a likewise very particularly preferred embodiment, the xanthene dyes used are ones of the formula (II-J)

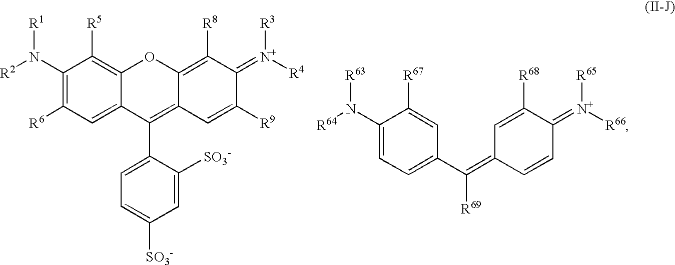

where

R¹ to R⁴ and R⁶³ to R⁶⁶ represent, independently of one another, hydrogen, methyl, ethyl, propyl or butyl or

NR¹R², NR³R⁴, NR⁶³R⁶⁴ and NR⁶⁵R⁶⁶ represent, independently of one another, pyrrolidino, piperidino or morpholino,

R⁵, R⁶, R⁸, R⁹, R⁶⁷ and R⁶⁸ represent, independently of one another, hydrogen or methyl or

R¹;R⁵, R²;R⁶, R³;R⁸, R⁴;R⁹, R⁶³;R⁶⁷ and R⁶⁵;R⁶⁸ represent, independently of one another, a —CH₂CH₂CH₂— bridge,

R⁶⁹ represents hydrogen, phenyl, 2-chlorophenyl, 4-dimethylaminophenyl, 4-diethylaminophenyl, 4-anilinophenyl, naphthyl, 4-dimethylaminonaphthyl or 4-anilinonaphthyl.

In a likewise very particularly preferred embodiment, the xanthene dyes used are ones of the formula (II-K)

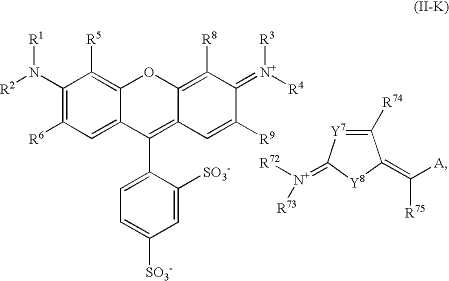

where

R¹ to R⁴, R⁷², R⁷³, R^{72′} and R^{73′} represent, independently of one another, hydrogen, methyl, ethyl, propyl or butyl, where R⁷² and R^{72′} or R⁷³ and R^{73′} are identical, or

NR¹R², NR³R⁴, NR⁷²R⁷³ and NR^{72′}R^{73′} represent, independently of one another, pyrrolidino, piperidino or morpholino, where NR⁷²R⁷³ and NR^{72′} R^{73′} are identical,

R⁵, R⁶, R⁸ and R⁹ represent, independently of one another, hydrogen or methyl or

R¹;R⁵, R²;R⁶, R³;R⁸ and R⁴;R⁹ represent, independently of one another, a —CH₂CH₂CH₂— bridge,

Y⁷ and Y^{7′} are identical and represent N or CH,

Y⁸ and Y^{8′} are identical and represent S,

R⁷⁴ and R^{74′} are identical and represent hydrogen, methyl, ethyl, propyl, butyl or phenyl,

R⁷⁵ represents hydrogen, phenyl, 4-dimethylaminophenyl or 4-diethylaminophenyl and

A represents 4-dimethylaminophenyl, 4-diethylaminophenyl, 4-N-methyl-cyanoethylaminophenyl, 4-N-methyl-hydroxyethylaminophenyl or a radical of the formula

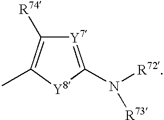

In a likewise very particularly preferred embodiment, the xanthene dyes used are ones of the formula (II-L)

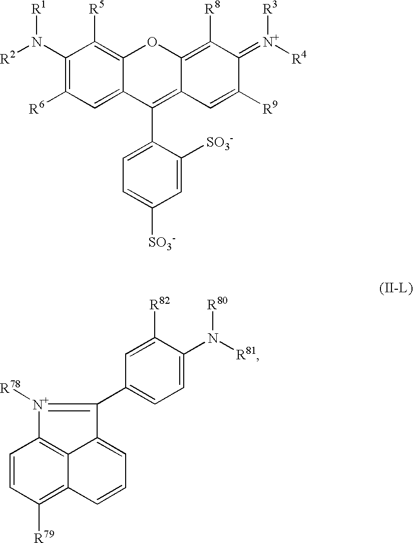

where

R¹ to R⁴, R⁸⁰ and R⁸¹ represent, independently of one another, hydrogen, methyl, ethyl, propyl or butyl or

NR¹R², NR³R⁴ and NR⁸⁰R⁸¹ represent, independently of one another, pyrrolidino, piperidino or morpholino,

R⁷⁸ represents methyl, ethyl, benzyl, cyanoethyl or hydroxyethyl,

R⁵, R⁶, R⁸, R⁹ and R⁸² represent, independently of one another, hydrogen or methyl or

R¹;R⁵, R²;R⁶, R³;R⁸, R⁴;R⁹ and R⁸⁰;R⁸² represent, independently of one another, a —CH₂CH₂CH₂— bridge, where the bridge R⁸⁰;R⁸² may be substituted by from 1 to 3 methyl groups, and

R⁷⁹ represents hydrogen or bromine.

In the case of a write-once optical data carrier according to the invention which is written on and read by means of the light of a blue laser, preference is given to xanthene dyes whose absorption maximum λₘₐₓ₂ is in the range from 420 to 550 nm, where the wavelength λ_{1/2} at which the absorbance in the short wavelength flank of the absorption maximum at the wavelength λₘₐₓ₂ is half of the absorbance value at λₘₐₓ₂ and the wavelength λ_{1/10} at which the absorbance in the short wavelength flank of the absorption maximum at the wavelength λₘₐₓ₂ is one tenth of the absorbance value at λₘₐₓ₂ are preferably not more than 50 nm apart. Such a xanthene dye preferably has no shorter-wavelength maximum λₘₐₓ₁ down to a wavelength of 350 nm, particularly preferably 320 nm, very particularly preferably 290 nm.

Preference is given to xanthene dyes having an absorption maximum λₘₐₓ of from 410 to 530 nm.

Particular preference is given to xanthene dyes having an absorption maximum λₘₐₓ₂ of from 420 to 510 nm.

Very particular preference is given to xanthene dyes having an absorption maximum λₘₐₓ₂ of from 430 to 500 nm.

In the case of these xanthene dyes, λ_{1/2} and λ_{1/10}, as defined above, are preferably not more than 40 nm apart, particularly preferably not more than 30 nm apart, very particularly preferably not more than 20 nm apart.

In the case of a write-once optical data carrier according to the invention which is written on and read by means of the light of a red laser, preference is also given to xanthene dyes whose absorption maximum λₘₐₓ₂ is in the range from 500 to 650 nm, where the wavelength λ_{1/2} at which the absorbance in the long wavelength flank of the absorption maximum at the wavelength λₘₐₓ₂ is half of the absorbance value at λₘₐₓ₂ and the wavelength λ_{1/10} at which the absorbance in the long wavelength flank of the absorption maximum at the wavelength λₘₐₓ₂ is one tenth of the absorbance value at λₘₐₓ₂ are preferably not more than 50 run apart. Such a xanthene dye preferably has no longer-wavelength maximum λₘₐₓ₃ up to a wavelength of 750 nm, particularly preferably up to 800 nm, very particularly preferably up to 850 nm.

Preference is given to xanthene dyes having an absorption maximum max of from 530 to 630 nm.

Particular preference is given to xanthene dyes having an absorption maximum λₘₐₓ₂ of from 550 to 620 nm.

Very particular preference is given to xanthene dyes having an absorption maximum λₘₐₓ₂ λₘₐₓ₂ of from 580 to 610 nm.

In the case of these xanthene dyes, λ_{1/2} and λ_{1/10}, as defined above, are preferably not more than 40 nm apart, particularly preferably not more than 30 nm apart, very particularly preferably not more than 20 nm apart.

The xanthene dyes have a molar extinction coefficient ε of >40 000 l/mol cm, preferably >60 000 l/mol cm, particularly preferably >80 000 l/mol cm, very particularly preferably >100 000 l/mol cm, at the absorption maximum λₘₐₓ₂.

The absorption spectra are, for example, measured in solution.

Some xanthene dyes of the formula (I) with cations other than those employed according to the invention are known.

The invention further provides xanthene dyes of the formula (I) in which the substituents have the abovementioned general, particular and very particular meanings and the abovementioned provisos apply.

The xanthene dyes of the formula (I) are prepared, for example, by reacting

xanthene dyes of the formula (I), in which M⁺ represents a cation which is not according to the invention M^{+′}, for example an alkali metal ion such as Li⁺, Na⁺, K⁺, a proton H⁺or an ammonium ion such as NH₄⁺, trimethylammonium or tetramethylammonium,

with salts M⁺Z⁻, where M⁺ is as defined above according to the invention and Z⁻ represents an anion, for example chloride, bromide, hydrogensulphate, ½ sulphate, methosulphate, acetate or tetrafluoroborate,

in a suitable solvent in which the starting materials of the formula (I) with M⁺=M^{+′} and M⁺Z⁻ preferably dissolve at least partly and the novel product of the formula (I) has a lower solubility. It can, for example, be isolated from the solvent by filtration with suction. Suitable solvents are, for example, water, alcohols such as methanol, ethanol, propanol, methoxyethanol, methoxypropanol, nitrites such as acetonitrile, amides such as dimethylformamide, N-methylpyrrolidone and esters such as γ-butyrolactone or mixtures thereof.

Another method of preparing the novel xanthene dyes of the formula (I) comprises reacting xanthene dyes of the formula (I) in which M⁺ is a cation which is not according to the invention M^{+′}, for example an alkali metal ion such as Li⁺, Na⁺, K⁺, a proton H⁺or an ammonium ion such as NH₄⁺, trimethylammonium or tetramethylammonium, with cation exchangers laden with cations according to the invention M⁺. Here too, suitable solvents are those described above. This method is advantageous when the novel xanthene dyes of the formula (I) are readily soluble in the solvent chosen. They are then isolated, for example, by taking off the solvent or by precipitation using a solvent in which they are sparingly soluble. Such solvents can be, for example, aromatics such as toluene or esters such as ethyl acetate.

The light-absorbent compounds described guarantee a sufficiently high reflectivity (>10%) of the optical data carrier in the unwritten state and a sufficiently high absorption for thermal degradation of the information layer on point-wise illumination with focused light if the wavelength of the light is in the range from 360 to 460 nm and from 600 to 680 nm. The contrast between written and unwritten points on the data carrier is achieved by the reflectivity change of the amplitude and also the phase of the incident light due to the changed optical properties of the information layer after the thermal degradation.

The xanthene dyes are preferably applied to the optical data carrier by spin coating. The xanthene dyes can be mixed with one another or with other dyes having similar spectral properties. In particular, dyes containing different cations can also be mixed. The information layer can comprise not only the xanthene dyes but also additives such as binders, wetting agents, stabilizers, diluents and sensitizers and also further constituents.

Apart from the information layer, further layers such as metal layers, dielectric layers, and protective layers may be present in the optical data carrier. Metals and dielectric layers serve, inter alia, to adjust the reflectivity and the heat absorption/retention. Metals can be, depending on the laser wavelength, gold, silver, aluminium, etc. Examples of dielectric layers are silicon dioxide and silicon nitride. Protective layers are, for example, photocurable surface coatings, (pressure-sensitive) adhesive layers and protective films.

Pressure-sensitive adhesive layers consist mainly of acrylic adhesives. Nitto Denko DA-8320 or DA-8310, disclosed in the patent JP-A 11-273147, can, for example, be used for this purpose.

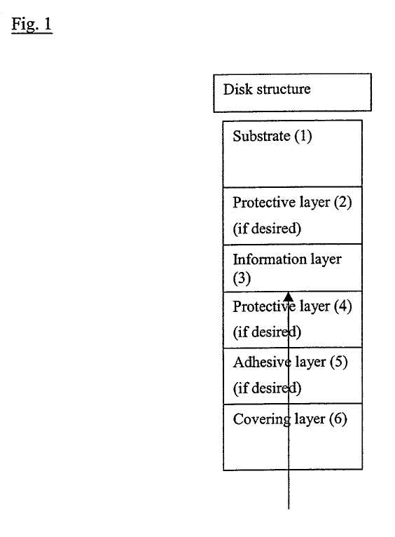

The optical data carrier has, for example, the following layer structure (cf. FIG. 1): a transparent substrate (1), if desired a protective layer (2), an information layer (3), if desired a protective layer (4), if desired an adhesive layer (5), a covering layer (6).

The structure of the optical data carrier preferably:

comprises a preferably transparent substrate (1) to whose surface at least one light-writeable information layer (3) which can be written on by means of light, preferably laser light, if desired a protective layer (4), if desired an adhesive layer (5) and a transparent covering layer (6) have been applied.

comprises a preferably transparent substrate (1) to whose surface a protective layer (2), at least one information layer (3) which can be written on by means of light, preferably laser light, if desired an adhesive layer (5) and a transparent covering layer (6) have been applied.

comprises a preferably transparent substrate (1) to whose surface a protective layer (2) if desired, at least one information layer (3) which can be written on by means of light, preferably laser light, if desired a protective layer (4), if desired an adhesive layer (5) and a transparent covering layer (6) have been applied.

comprises a preferably transparent substrate (1) to whose surface at least one information layer (3) which can be written on by means of light, preferably laser light, if desired an adhesive layer (5) and a transparent covering layer (6) have been applied.

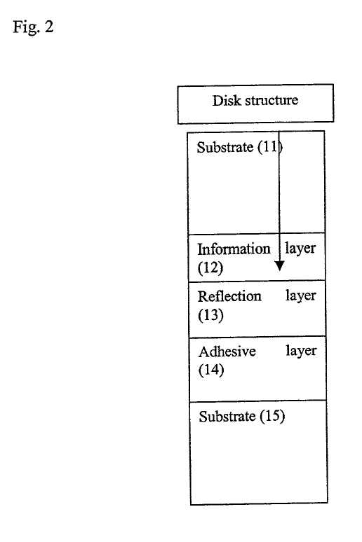

Alternatively, the optical data carrier has, for example, the following layer structure (cf. FIG. 2): a preferably transparent substrate (11), an information layer (12), if desired a reflection layer (13), if desired an adhesive layer (14), a further preferably transparent substrate (15).

The invention further provides optical data carriers according to the invention which have been written on by means of blue or red light, in particular laser light.

The following examples illustrate the subject matter of the invention.

### EXAMPLES

### Example 1

6.3 g of the xanthene dye of the formula

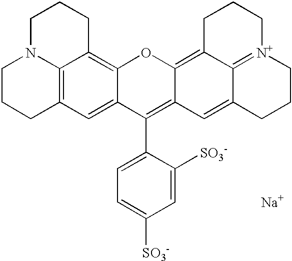

(Rhodamin 660) were dissolved in 200 ml of water. 2.8 g of ferrocenyl tetrafluoroborate were slowly sprinkled in at room temperature while stirring. The mixture was stirred overnight at room temperature and filtered with suction through a G4 frit. This gave 4.5 g (57% of theory) of a shining golden powder of the formula

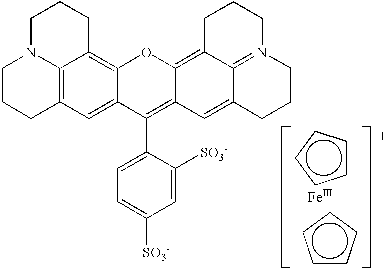

m.p. >300° C.

λₘₐₓ(methanol)=578 nm

ε=121217 l/mol cm

Solubility: >2% in TFP (2,2,3,3-tetrafluoropropanol)

Further suitable xanthene dyes are shown in the table; these can be prepared by methods analogous to Example 1:

| Example | 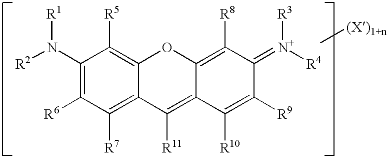 | M⁺ | λₘₐₓ/nm¹⁾ | ε/1/mol cm |
|---|---|---|---|---|
| 2 | 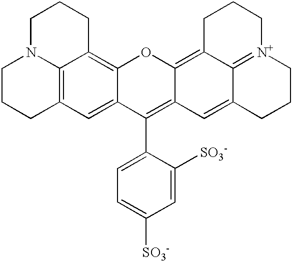 | 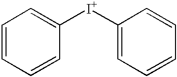 | 577 | 122100 |
| 3 | 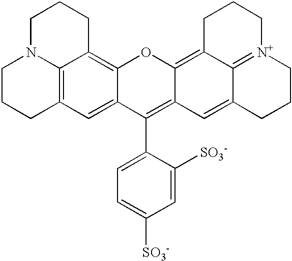 | 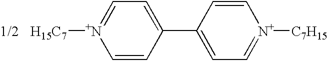 | 578 | 234350 |
| 4 | 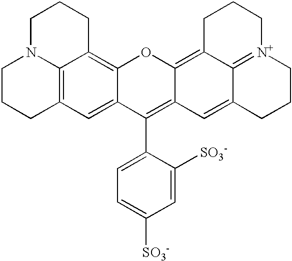 | 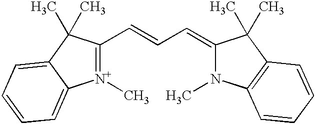 | 544/577 | 118058 at 577 nm |
| 5 | 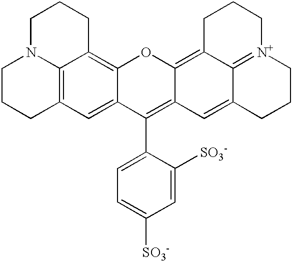 | 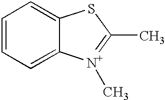 | 578 | |
| 6 | 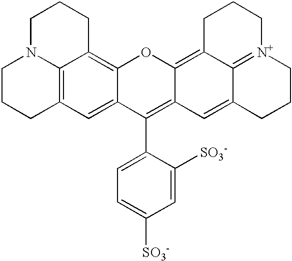 | 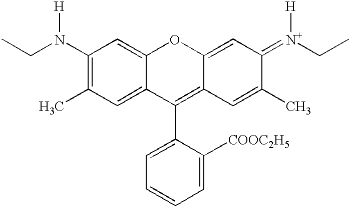 | 521/578 | |
| 7 | 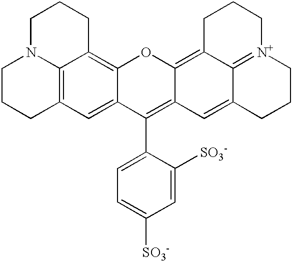 | 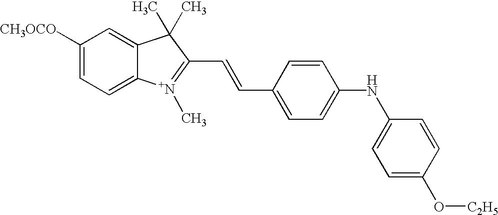 | | |
| 8 | 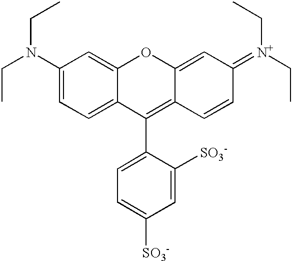 | 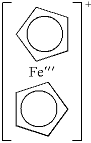 | 555 | 103060 |
| 9 | 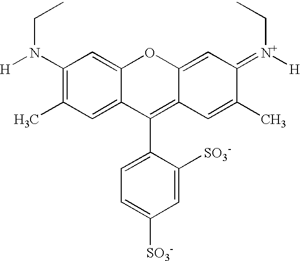 | 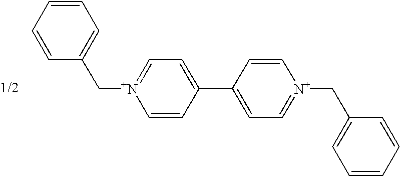 | 528 | |
| 10 | 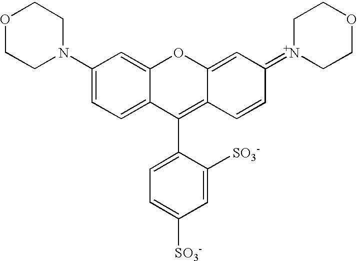 | 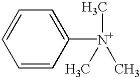 | | |
| 11 | 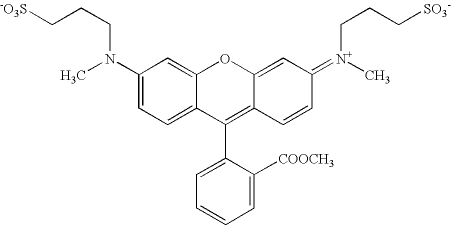 | 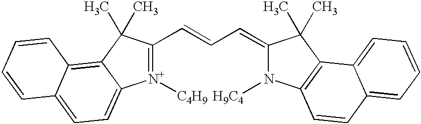 | | |
| 12 | 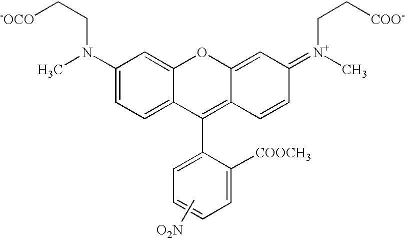 | 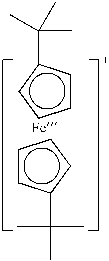 | | |
| 13 | 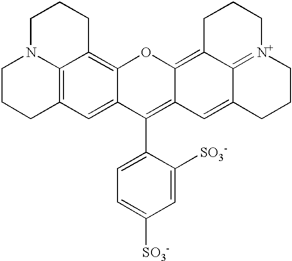 | 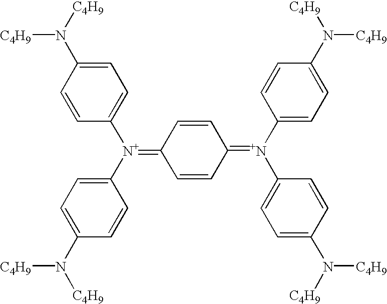 | 578, 964 | |
| 14 | 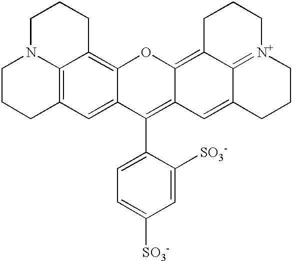 | 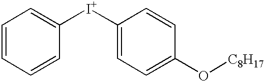 | 578 | |
| 15 | 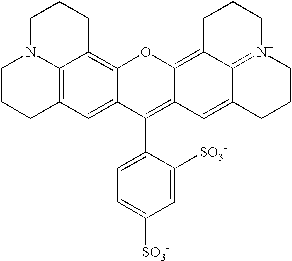 | 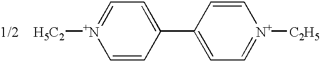 | 577 | 247850 |
| 16 | 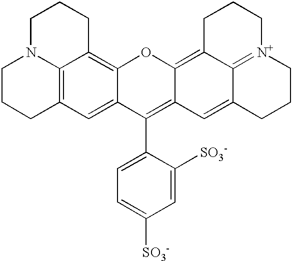 | 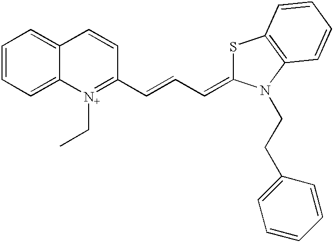 | 578 | 246220 |
| 17 | 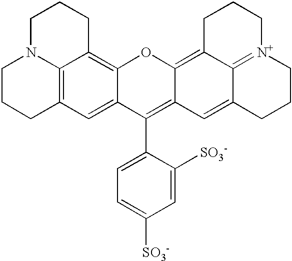 | 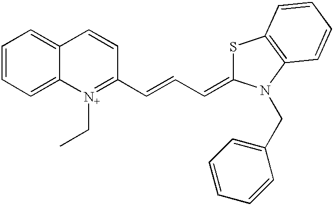 | 577 | |
| 18 | 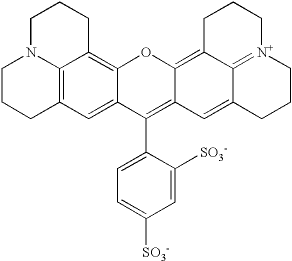 | 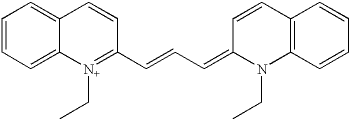 | 579, 600 | 182728 at 579 nm |
| 19 | 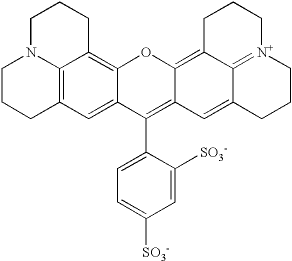 | 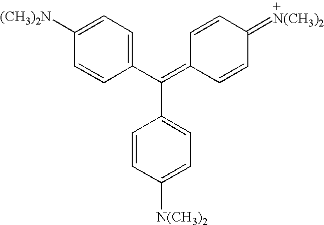 | 578, 595 | |
| 20 | 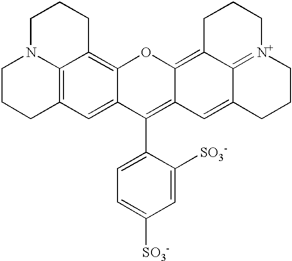 | 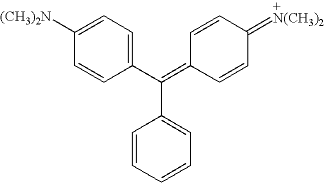 | 578, 605 | |
| 21 | 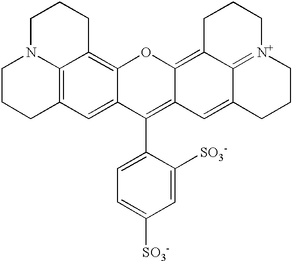 | 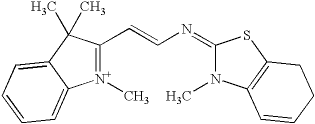 | 549, 577 | 125050 |
| 22 | 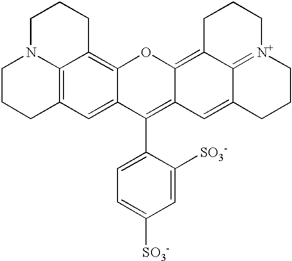 | 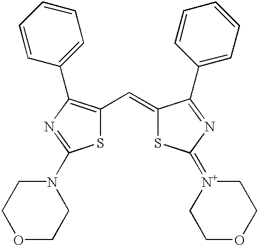 | 580 | 102100 |
| 23 | 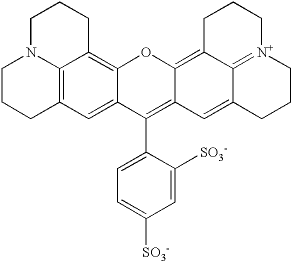 | 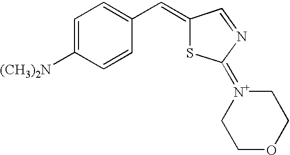 | 577 | 120160 |
| 24 | 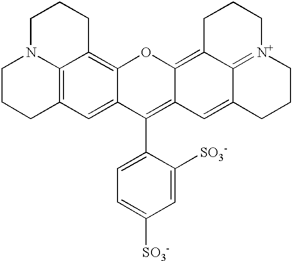 | 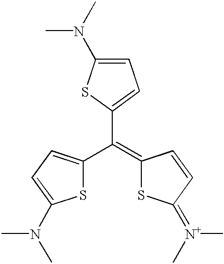 | 578, 607 | |
| 25 | 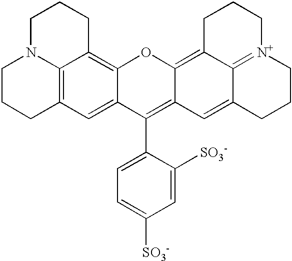 | 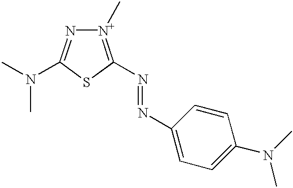 | 578, 599 | |
| 26 | 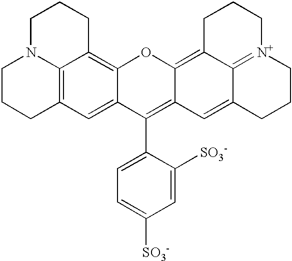 | 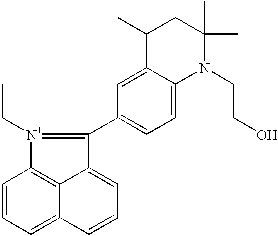 | 576 | |
| 27 | 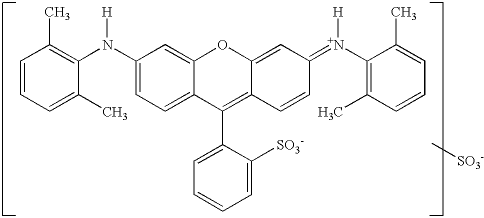 | 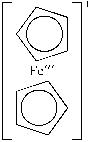 | 527 | |
| 28 | 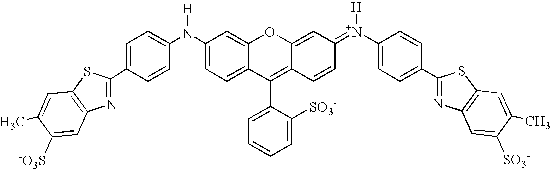 | 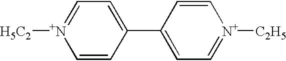 | 587 | |
| 29 | 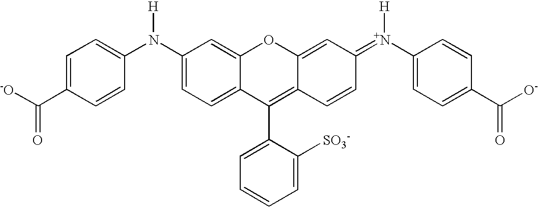 | 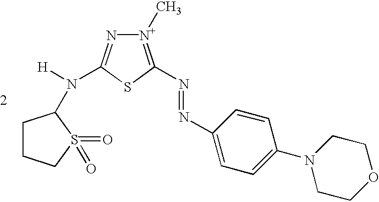 | 602 | |
| 30 | 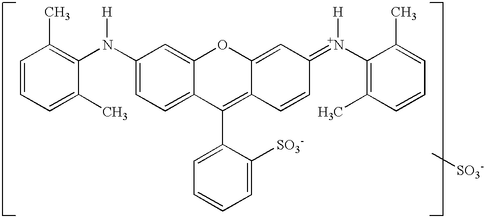 | 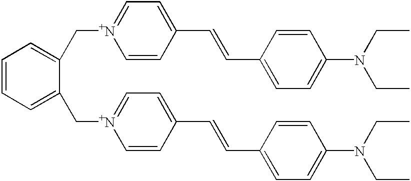 | 503/527 | |
| 31 | 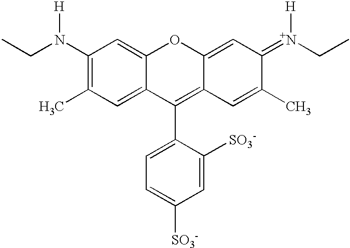 | 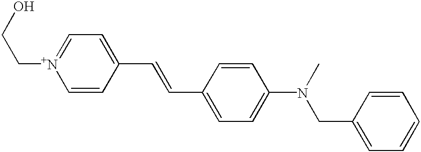 | 482/528 | |
| 32 | 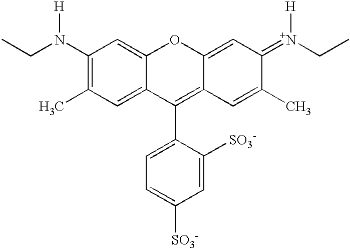 | 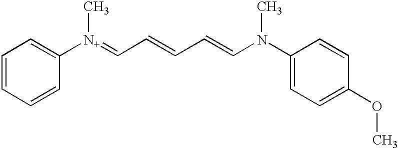 | 450/528 | |
| 33 | 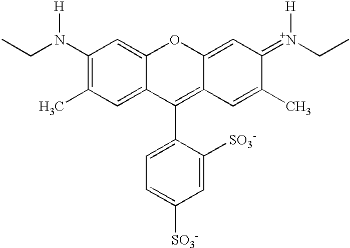 | 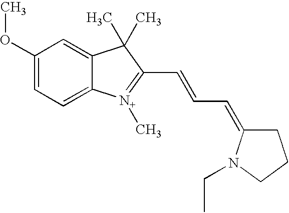 | 438/528 | |
| 34 | 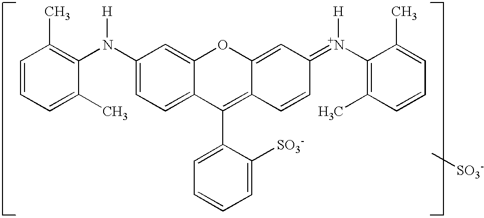 | 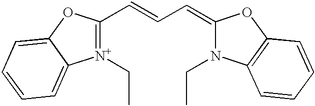 | 482/527 | |
| 35 | 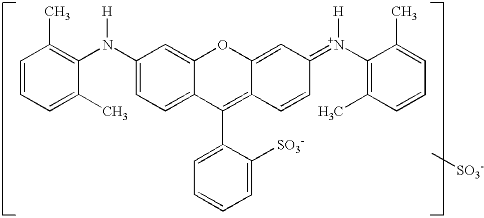 | 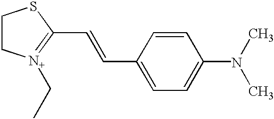 | 478/527 | |
| 36 | 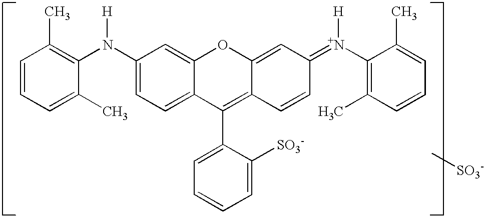 | 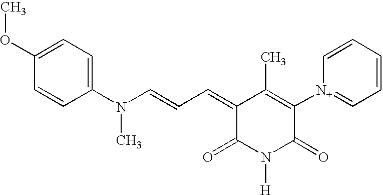 | 469/527 | |
| 37 | 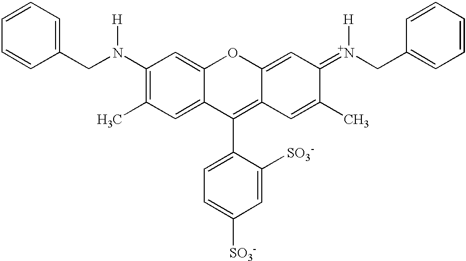 | 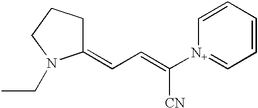 | 462/515 | |
| 38 | 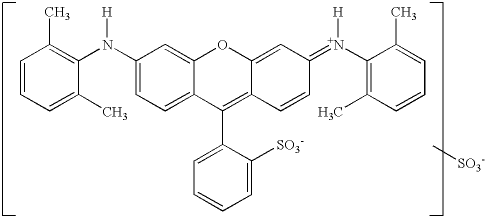 | 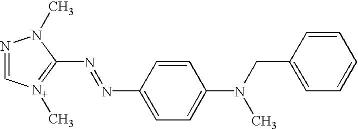 | 527 | |
| 39 | 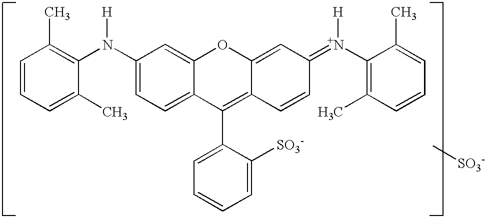 | 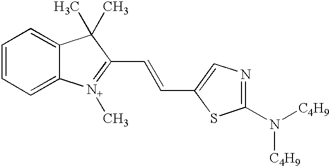 | 528 | |

¹⁾ in methanol, unless indicated otherwise.

### Example 40

A 4% strength solution of the dye from Example 15 in 2,2,3,3-tetrafluoropropanol was prepared at room temperature. This solution was applied by means of spin coating to a pregrooved polycarbonate substrate. The pregrooved polyearbonate substrate had been produced as a disk by means of injection moulding. The dimensions of the disk and the groove structure corresponded to those customarily used for DVD-Rs. The disk with the dye layer as information carrier was coated with 100 nm of silver by vapour deposition. A UV-curable acrylic coating composition was subsequently applied by spin coating and cured by means of a UV lamp. The disk was tested by means of a dynamic writing test apparatus constructed on an optical tester bench comprising a diode laser (λ=656 nm) for generating linearly polarized light, a polarization-sensitive beam splitter, a λ/4 plate and a moveably suspended collecting lens having a numerical aperture NA=0.6 (actuator lens). The light reflected from the reflection layer of the disk was taken out from the beam path by means of the abovementioned polarization-sensitive beam splitter and focused by means of an astigmatic lens onto a four-quadrant detector. At a linear velocity V=3.5 m/s and a writing power P_{W}=15 mW, a signal/noise ratio C/N=52 dB was measured. The writing power was applied as an oscillating pulse sequence, with the disk being irradiated alternately for 1 μs with the abovementioned writing power P_{W} and for 4 μs with the reading power Pᵣ˜0.6 mW. The disk was irradiated with this oscillating pulse sequence until it had rotated once. The marking produced in this way was then read using the reading power Pᵣ and the abovementioned signal/noise ratio C/N was measured.

1. Optical data carrier comprising a preferably transparent substrate which may, if desired, have previously been coated with one or more reflection layers and to whose surface a light-writeable information layer, if desired one or more reflection layers and, if desired, a protective layer or a further substrate or a covering layer have been applied, which can be written on or read by means of blue or red light, preferably laser light, where the information layer comprises a light-absorbent compound and, if desired, a binder, characterized in that at least one xanthene dye which contains at least two anionic groups and has at least one cation containing at least one conjugated a system having at least 6 π electrons as counterion, with the proviso that the cation is not benzyltrimethylammonium, benzyltriethylammoniurn, tetraphenylphosphonium, butyltriphenylphosphonium or ethyltriphenylphosphonium, is used as light-absorbent compound.

2. Optical data carrier according to claim 1, characterized in that the xanthene dye has the formula (I) 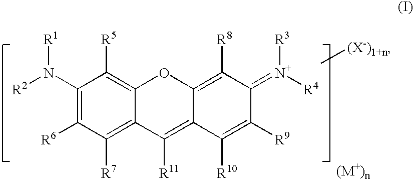
where
R¹ to R⁴ represent, independently of one another, hydrogen, C₁-C₁₆-alkyl, C₃-C₆-alkenyl, C₅-C₇-cycloalkyl, C₇-C₁₆-aralkyl, C₆-C₁₀-aryl or a heterocyclic radical, which may be substituted by nonionic radicals or an anionic group X⁻ or
NR¹R² or NR³R⁴ represent, independently of one another, a five- or six-membered saturated ring which is bound via N and may additionally contain an N or O atom and/or be substituted by nonionic radicals,
R⁵ to R¹⁰ represent, independently of one another, hydrogen, halogen, C₁-C₁₆-alkyl, C₁-C₁₆-alkoxy, C₁-C₁₆-alkylthio, cyano or nitro or
R¹;R⁵, R²;R⁶, R³;R⁸ or R⁴;R⁹ represent, independently of one another, a two- or three-membered bridge which may contain an N or O atom and/or be substituted by nonionic radicals,
R¹¹ represents hydrogen, C₁-C₁₆-alkyl, C₅-C₇-cycloalkyl, C₆-C₁₀-aryl or a heterocyclic radical which may be substituted by nonionic radicals or an anionic group X—,
X⁻ represents an anionic group of the formula —COO⁻, —SO₃⁻ or —O—SO₃⁻ or one equivalent of a dianionic group of the formula —PO₃²⁻ or —O—PO₃²⁻,
M⁺ represents a cation or one equivalent of a polycation which contains at least one conjugated π system having at least 6 π electrons, and
n represents an integer from 1 to 3,
with the proviso that M⁺ does not represent benzyltrimethylammonium, benzyltriethylammonium, tetraphenylphosphonium, butyltriphenylphosphonium or ethyltriphenylphosphonium.

3. Optical data carrier according to claim 1 or 2, characterized in that
M⁺ represents
a) an aromatically or heteroaromatically substituted ammonium, sulphonium or iodonium salt,
b) a cyclic onium salt,
c) a redox system in its oxidized cationic or radical-cationic form,
d) a cationic dye system.

4. Optical data carrier according to one or more of claims 1 to 3, characterized in that, in formula (I)
R¹ to R⁴ represent, independently of one another, hydrogen, methyl, ethyl, propyl, butyl, chloroethyl, cyanoethyl, hydroxyethyl, hydroxypropyl, —CH₂CH₂COO^{—}, —CH₂CH₂CH₂COO^{—}, —CH₂CH₂CH₂CH₂COO^{—}, —CH₂CH₂SO₃⁻, —CH₂CH₂CH₂SO₃⁻, —CH₂CH₂CH₂CH₂SO₃⁻, —CH₂CH₂OSO₃⁻, allyl, cyclopentyl, cyclohexyl, benzyl, phenethyl, phenyl, tolyl, anisyl, —C₆H₄—SO₃⁻, pyridyl or furyl or
NR¹R² or NR³R⁴ represent, independently of one another, pyrrolidino, piperidino, morpholino, piperazino or N-methylpiperazino,
R⁵ to R¹⁰ represent, independently of one another, hydrogen, chlorine, methyl or methoxy or
R¹;R⁵, R²;R⁶, R³;R⁸ or R⁴;R⁹ represent, independently of one another, a —CH₂CH₂—, —CH₂CH₂CH₂— or —CH₂CH₂—O— bridge,
R¹¹ represents hydrogen, —CH₂CH₂COO^{—}, —CH₂CH₂CH₂COO^{—}, —CH₂CH₂CH₂CH₂COO^{—}, —CH₂CH₂SO₃⁻, —CH₂CH₂CH₂SO₃⁻, —CH₂CH₂CH₂CH₂SO₃⁻, —CH₂CH₂OSO₃⁻, phenyl, naphthyl or pyridyl which are substituted by up to two —COO^{—}, —SO₃⁻, CN, —COO-methyl to -butyl radicals,
where the radicals R¹ to R⁴ and R¹¹ contain a total of at least two —COO⁻ or —SO₃⁻ groups,
M⁺ represents a cation or one equivalent of a polycation of one of the following formulae, 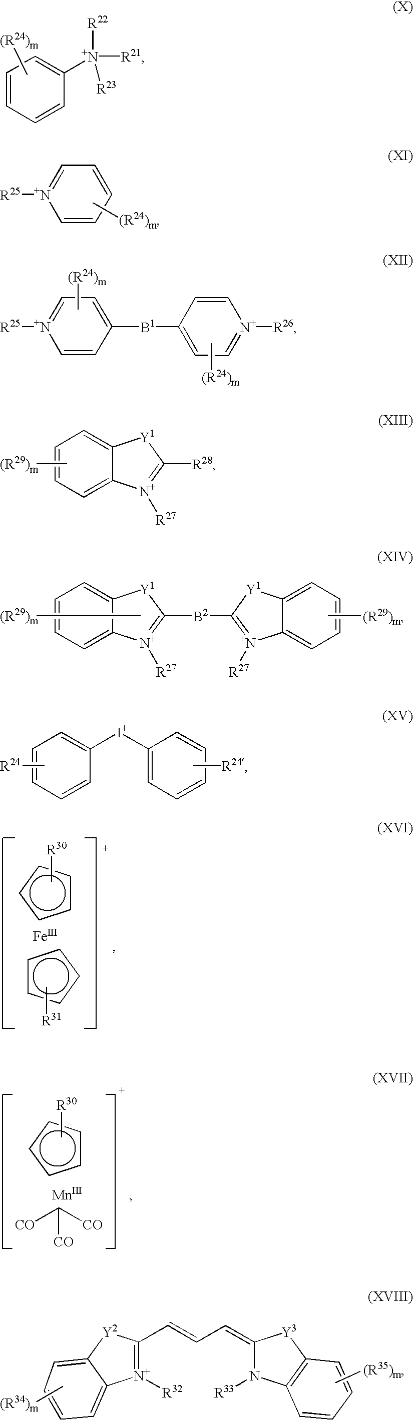
where
R²¹ to R²³, R³⁶, R³⁷, R³⁹ to R⁴², R⁵¹ to R⁵⁴, R⁵⁷, R⁶¹ to R⁶⁶, R⁷², R⁷³, R^{72′}, R^{73′}, R⁷⁶, R⁷⁷, R⁸⁰ and R⁸¹ represent, independently of one another, hydrogen, C₁-C₁₆-alkyl, C₃-C₆-alkenyl, C₅-C₇-cycloalkyl, C₇-C₁₆-aralkyl or C₆-C₁₀-aryl which may be substituted by nonionic radicals or
two adjacent radicals together with the nitrogen atom connecting them represent, independently of one another, a five- or six-membered saturated ring which is bound via N and may additionally contain an N or O atom and/or be substituted by nonionic radicals,
R²⁵ to R²⁷, R³², R³³ and R⁷⁸ represent, independently of one another, C₁-C₁₆-alkyl, C₃-C₆-alkenyl, C₅-C₇-cycloalkyl, C₇-C₁₆-aralkyl or C₆-C₁₀-aryl which may be substituted by nonionic radicals,
R²⁸ represents hydrogen, chlorine, amino, C₁-C₁₆-alkyl, C₃-C₆-alkenyl, C₅-C₇-cycloalkyl, C₇-C₁₆-aralkyl or C₆-C₁₀-aryl,
R²⁴, R^{24′}, R²⁹ to R³¹, R³⁴, R³⁵ and R⁷⁹ represent, independently of one another, hydrogen, halogen, C₁-C₈-alkyl, C₁-C₈-alkoxy, C₁-C₄-alkylthio, cyano or nitro or
two adjacent radicals R²⁴, R²⁹, R³⁴ and R³⁵ represent a —CH═CH—CH═CH— bridge,
R³⁸, R⁵⁵ and R⁵⁶ represent, independently of one another, hydrogen, halogen, C₁-C₄-alkyl, C₁-C₄-alkoxy, cyano, nitro, C₁-C₄-alkoxycarbonyl, C₁-C₄-alkanoylamino or C₁-C₄-alkanesulphonylamino and R³⁸ together with R³⁶ may form a —(CH₂)₂— or —(CH₂)₃— bridge,
R⁴³ to R⁴⁸, R⁶⁰, R⁶⁷, R⁶⁸ and R⁸² represent, independently of one another, hydrogen, halogen, C₁-C₈-alkyl, C₁-C₈-alkoxy or C₁-C₄-alkylthio and R⁴³ together with R³⁹, R⁴⁴ together with R⁴⁰, R⁴⁶ together with R⁴¹, R⁴⁷ together with R⁴¹, R⁶⁷ together with R⁶³, R⁶⁸ together with R⁶⁵ and R⁸² together with R⁸⁰ may form a —(CH₂)₂— or —(CH₂)₃— bridge,
R⁴⁹, R⁷⁴ and R^{74′} represent, independently of one another, hydrogen, C₁-C₁₆-alkyl, C₅-C₇-cycloalkyl or C₆-C₁₀-aryl which may be substituted by nonionic radicals,
Y¹ to Y³ represent, independently of one another, O, S, NR⁵⁷, CR⁵⁸R⁵⁹ or —CH═CH—,
Y⁴ represents CR⁶⁰ or N,
Y⁵ and Y⁶ represent, independently of one another, O, S, NR⁵⁷ or CR⁵⁸R⁵⁹,
Z, Y⁷ and Y^{7′} represent, independently of one another, N, CH or C—CN,
Y⁸ and Y^{8′} represent, independently of one another, O or S,
R⁵⁸ and R⁵⁹ represent, independently of one another, hydrogen or C₁-C₄-alkyl or
CR⁵⁸R⁵⁹ represents a ring of the formula 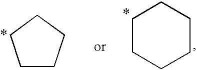
where two single bonds go out from the asterisked (*) atom,
R⁵⁰ represents hydrogen, halogen, C₁-C₄-alkyl, C₁-C₄-alkoxy, C₁-C₄-alkylthio, cyano, nitro, C₁-C₄-alkoxycarbonyl, mono- or dialkylamino, pyrrolidino, piperidino or morpholino or
R⁵⁰; R⁶⁰ form a —CH═CH—CH═CH— bridge,
R⁶⁹ and R⁷⁵ represent, independently of one another, hydrogen, C₁-C₄-alkyl or a radical of the formula 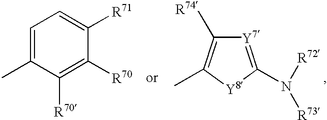
R⁷⁰ and R^{70′} represent, independently of one another, hydrogen, halogen, C₁-C₈-alkyl, C₁-C₈-alkoxy or C₁-C₄-alkylthio or together form a —CH═CH—CH═CH— bridge or R⁷⁰ together with R⁷⁷ may form a —(CH₂)₂— or —(CH₂)₃— bridge,
R⁷¹ represents hydrogen, halogen, C₁-C₈-alkyl, C₁-C₈-alkoxy, C₁-C₄-alkylthio, mono- or di-C₁-C₈-alkylamino, anilino or N—C₁-Cg-alkyl-anilino,
A represents a radical of the formula 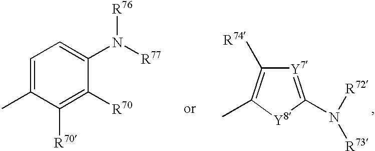
B¹ represents a direct bond, —CH═CH— or —C≡C—,
B² represents a direct bond, —CH═CH—, —C≡C— or thien-2,5-diyl,
Het represents a five- or six-membered aromatic or pseudoaromatic heterocyclic ring which contains from 1 to 3 heteroatoms selected from the group consisting of N, O and S and may be benzo-fused and/or substituted by up to three nonionic radicals,
m represents an integer from 1 to 3, where, if m>1, the radicals indexed by m may have different meanings and
n represents an integer from 1 to 2.

5. Optical data carrier according to one or more of claims 1 to 4, characterized in that the xanthene dye has the formula (II), 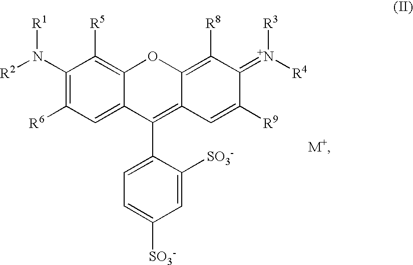
where
R¹ to R⁴ represent, independently of one another, hydrogen, methyl, ethyl, propyl, butyl, cyanoethyl, hydroxyethyl, hydroxypropyl, cyclohexyl, benzyl or phenyl or
NR¹R² or NR³R⁴ represent, independently of one another, pyrrolidino, piperidino or morpholino,
R⁵, R⁶, R⁸ and R⁹ represent, independently of one another, hydrogen, methyl or methoxy or
R¹;R⁵, R²;R⁶, R³;R⁸ or R⁴;R⁹ represent, independently of one another, a —CH₂CH₂CH₂— bridge,
M⁺ is a cation or one equivalent of a polycation of one of the formulae (X) to (XII), (XV), (XVI), (XVIII) to (XX), (XXIV), (XXVI), (XXVII) or (XXVIII),
where
R²¹ R²³, R³⁶, R³⁷, to R⁴², R⁵⁷, R⁶¹ R⁷², R⁷³, R^{72′}, R^{73′}, R⁷⁶, R⁷⁷, R⁸⁰ and R⁸¹ represent, independently of one another, hydrogen, methyl, ethyl, propyl, butyl, cyanoethyl, hydroxyethyl, hydroxypropyl, cyclohexyl, benzyl or phenyl or
NR²¹R²², NR³⁶R³⁷, NR³⁹R⁴⁰, NR⁴¹R⁴², NR⁶¹R⁶² and NR⁸⁰R⁸¹ represent, independently of one another, pyrrolidino, piperidino or morpholino,
R²⁵ to R²⁷, R³², R³³ and R⁷⁸ represent, independently of one another, methyl, ethyl, propyl, butyl, cyanoethyl, hydroxyethyl, hydroxypropyl, cyclohexyl or benzyl,
R²⁴, R^{24′}, R³⁴, R³⁵ and R⁷⁹ represent, independently of one another, hydrogen, chlorine, methyl, methoxy, cyano or nitro or two adjacent radicals R²⁴, R³⁴ or R³⁵ represent a —CH═CH—CH═CH-bridge,
R³⁰ and R³¹ are identical and represent methyl, ethyl, propyl, 2-propyl, butyl or tert-butyl,
R³⁸ represents hydrogen, chlorine, methyl, methoxy, cyano, nitro, methoxycarbonyl, acetylamino or methanesulphonylamino,
R⁴³ to R⁴⁸, R⁶⁷, R⁶⁸ and R⁸² represent, independently of one another, hydrogen, chlorine, methyl or methoxy,
R⁴⁹, R⁷⁴ and R^{74′} represent, independently of one another, methyl, cyclohexyl or phenyl,
B¹ represents a direct bond,
Y² and Y³ are identical and represent O, S, R⁵⁷, CR⁵⁸R⁵⁹ or —CH═CH—,
Y⁶ represents O, S or NR⁵⁷,
R⁵⁸ and R⁵⁹ are identical and represent methyl,
Z, Y⁷ and Y^{7′} represent CH,
Y⁸ and Y^{8′} represent O or S and are identical,
R⁶⁹ represents hydrogen or a radical of the formula 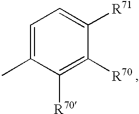
R⁷⁵ represents hydrogen or a radical of the formula 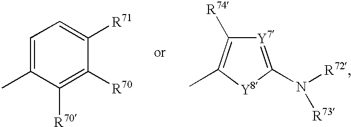
R⁷⁰ and R^{70′} represent, independently of one another, hydrogen, chlorine, methyl or methoxy or together form a —CH═CH—CH═CH— bridge,
R⁷¹ represents hydrogen, chlorine, methyl, methoxy, ethoxy, dimethylamino, diethylamino, N-methyl-N-cyanoethylamino, N-methyl-N-hydroxyethylamino, anilino or N-methyl-anilino,
A represents a radical of the formula 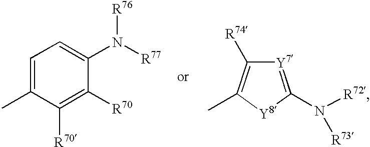
m represents an integer from 1 to 3, w here, if m>1, the radicals indexed by m may have different meanings.

6. Xanthene dyes of the formula (I) 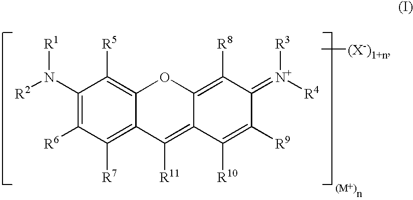
where
R¹ to R⁴ represent, independently of one another, hydrogen, C₁-C₁₆-alkyl, C₃-C₆-alkenyl, C₅-C₇-cycloalkyl, C₇-C₁₆-aralkyl, C₆-C₁₀-aryl or a heterocyclic radical, which may be substituted by nonionic radicals or an anionic group X⁻ or
NR¹R² or NR³R⁴ represent, independently of one another, a five- or six-membered saturated ring which is bound via N and may additionally contain an N or O atom and/or be substituted by nonionic radicals,
R⁵ to R¹⁰ represent, independently of one another, hydrogen, halogen, C₁-C₁₆-alkyl, C₁-C₁₆-alkoxy, C₁-C₁₆-alkylthio, cyano or nitro or
R¹;R⁵, R²;R⁶, R³;R⁸ or R⁴;R⁹ represent, independently of one another, a two- or three-membered bridge which may contain an N or O atom and/or be substituted by nonionic radicals,
R¹¹ represents hydrogen, C₁-C₁₆-alkyl, C₅-C₇-cycloalkyl, C₆-C₁₀-aryl or a heterocyclic radical which may be substituted by nonionic radicals or an anionic group X⁻,
X⁻ represents an anionic group of the formula —COO^{—}, —SO₃⁻ or —O—SO₃⁻ or one equivalent of a dianionic group of the formula —PO₃²⁻ or —O—P₃²⁻,
M⁺ represents a cation or one equivalent of a polycation which contains at least one conjugated n system having at least 6 π electrons, and
n represents an integer from 1 to 3,
with the proviso that M⁺ does not represent benzyltrimethylammonium, benzyltriethylammonium, tetraphenylphosphonium, butyltriphenylphosphonium or ethyltriphenylphosphonium.

7. Process for preparing xanthene dyes according to claim 6, characterized in that xanthene dyes of the formula (I) in which M⁺ has a meaning other than that specified in claim 6 are reacted with salts of the formula
M⊕Z⊖
where
M⊕ is as defined in claim 6 and
Z⊖ represents an anion in a solvent.

8. Use of xanthene dyes which contain at least two anionic groups and have at least one cation containing at least one conjugated π system having at least 6 π electrons as counterion, with the proviso that the cation is not benzyltrimethylammonium, benzyltriethylammonium, tetraphenylphosphonium, butyltriphenylphosphonium or ethyltriphenylphosphonium, in the information layer of write-once optical data carriers, where the xanthene dyes have an absorption maximum λₘₐₓ₂ in the range from 420 to 650 nm.

9. Use of xanthene dyes which contain at least two anionic groups and have at least one cation containing at least one conjugated π system having at least 6π electrons as counterion, with the proviso that the cation is not benzyltrimethylammonium, benzyltriethylammonium, tetraphenylphosphonium, butyltriphenylphosphonium or ethyltriphenylphosphonium, in the information layer of write-once optical data carriers, where the data carriers can be written on and read by means of blue or red, in particular red, laser light.

10. Process for producing the optical data carriers according to claim 1, which is characterized in that a preferably transparent substrate which may, if desired, have previously been coated with a reflection layer is coated with the xanthene dyes, if desired in combination with suitable binders and additives and, if desired, suitable solvents, and provided, if desired, with a reflection layer, further intermediate layers and, if desired, a protective layer or a further substrate or a covering layer.

11. Optical data carriers according to claim 1 which can be written on by means of blue or red, in particular red, light, in particular red laser light.
